(19) United States
(12) Patent Application Publication
(10) Pub. No.: US 2016/0100840 A1
(43) Pub. Date: Apr. 14, 2016

(54) METHODS, SYSTEMS AND DEVICES FOR TREATING ERECTILE DYSFUNCTION

(71) Applicant: Rox Medical, Inc., San Clemente, CA (US)

(72) Inventors: Rodney Brenneman, San Juan Capistrano, CA (US); Peter Balmforth, Oslo (NO); J. Christopher Flaherty, Auburndale, FL (US)

(21) Appl. No.: 14/970,217

(22) Filed: Dec. 15, 2015

Related U.S. Application Data
(63) Continuation of application No. PCT/US2014/044601, filed on Jun. 27, 2014.
(60) Provisional application No. 61/840,365, filed on Jun. 27, 2013.

(51) Int. Cl. A61B 17/11

(52) U.S. Cl. CPC A61B 17/11; A61B 2017/1107; A61B 2017/1139

(57) ABSTRACT

Provided are methods, systems and devices for treating erectile dysfunction in a patient. A patient is selected that exhibits erectile dysfunction. A flow pathway is created between a first vascular location comprising a source of arterial blood and a second vascular location comprising a source of venous blood.

### CROSS-REFERENCE TO RELATED APPLICATIONS

This application is a continuation of PCT Application No. PCT/US2014/044601 (Attorney Docket No. 29919-712.301), filed Jun. 27, 2014, which claims priority under 35 USC 119(e) to U.S. Provisional Patent Application Ser. No. 61/840,365, entitled “Methods, Systems and Devices for Treating Erectile Dysfunction”, filed Jun. 27, 2013, which is incorporated herein by reference by its entirety. This present application, while not claiming priority to, may be related to: U.S. Pat. No. 7,828,814, entitled “Device and Method for Establishing an Artificial Arterio-Venous Fistula”, filed Apr. 4, 2007; U.S. Non-Provisional application Ser. No. 11/152,621, entitled “Devices for Arterio-Venous Fistula Creation”, filed Jun. 13, 2005; U.S. Pat. No. 8,016,782, entitled “Methods for Providing Oxygenated blood to Venous Circulation”, filed Jun. 13, 2005; U.S. Non-Provisional application Ser. No. 11/946,454, entitled “Devices, Systems, and Methods for Creation of a Peripherally Located Fistula”, filed Nov. 28, 2007; U.S. Pat. No. 8,382,697, entitled “Devices, Systems, and Methods for Peripheral Arteriovenous Fistula Creation”, filed Jan. 22, 2008; U.S. Non-Provisional application Ser. No. 12/752,397, entitled “Device and Method for Establishing an Artificial Arteriovenous Fistula”, filed Apr. 1, 2010; U.S. Non-Provisional application Ser. No. 12/905,412, entitled “Devices, Systems, and Methods for Enhanced Visualization of the Anatomy of a Patient”, filed Oct. 15, 2010; the contents of each are incorporated by reference in their entirety.

### TECHNICAL FIELD

The embodiments disclosed herein relate generally to systems, devices and methods for treating a patient, particularly a patient afflicted with erectile dysfunction.

### BACKGROUND

Erectile dysfunction (impotence) occurs when a male cannot get or keep an erection firm enough for sexual intercourse. There can be both physical and psychological causes of erectile dysfunction. Some physical causes include heart disease, clogged blood vessels, high cholesterol, high blood pressure, diabetes, obesity, metabolic syndrome, Parkinson's disease, multiple sclerosis, low testosterone, Peyronie's disease (development of scar tissue inside the penis), certain prescription medications, tobacco use, substance abuse, treatments for prostate cancer or enlarged prostate, and surgeries or injuries that affect the pelvic area or spinal cord.

Complications resulting from erectile dysfunction can include an unsatisfactory sex life, stress or anxiety, embarrassment or low self-esteem, marital or relationship problems, and the inability to procreate.

Current treatment methods, such as the administration of pharmaceuticals and procedures such as implantation of penis pumps or other invasive implants, are associated with incomplete or otherwise limited treatment, high cost, invasiveness, and numerous undesirable side effects. There is therefore a need for improved approaches, including systems, devices and methods, for treating patients exhibiting erectile dysfunction.

### SUMMARY

According to one aspect of the present inventive concepts, a method of treating erectile dysfunction in a patient comprises selecting a patient exhibiting erectile dysfunction and creating a flow pathway between a first vascular location and a second vascular location. The first vascular location comprises a source of arterial blood and the second vascular location comprises a source of venous blood. The method is constructed and arranged to treat the patient's erectile dysfunction.

In some embodiments, the method is further constructed and arranged to restrict venous outflow from the penis.

In some embodiments, the method is further constructed and arranged to cause an increase in penile blood pressure, such as an increase in penile venous blood pressure.

In some embodiments, the method is further constructed and arranged to cause a physiologic change in the patient selected from the group consisting of: increased penile blood flow; decreased venous drainage; decreased venous outflow; increased penile blood pressure; increased oxygen delivery by the arterial system; increased blood volume; increased proportion of blood flow to the descending aorta; increased blood flow to the kidneys; increased blood flow outside the kidneys; increased cardiac output; and combinations thereof.

In some embodiments, the method is further constructed and arranged to reduce arterial hypertension. In these embodiments, the method can be further constructed and arranged to increase local penile venous pressure.

In some embodiments, the method is further constructed and arranged to cause at least one of an increase in oxygenation or an increase in flow rates associated with the patient's chemo-receptors.

In some embodiments, the flow pathway comprises a fistula.

In some embodiments, the flow pathway comprises an anatomical location relatively proximate to and/or within the penis of the patient.

In some embodiments, the first vascular location comprises an artery selected from the group consisting of: aorta; axillary; brachial; ulnar; radial; profundal; femoral; iliac; popliteal; internal pudendal; bulbourethral; dorsal; cavernosal; and carotid. In these embodiments, the second vascular location can comprise a vein.

In some embodiments, the second vascular location comprises a vein selected from the group consisting of: inferior vena cava; saphenous; femoral; iliac; popliteal; brachial; basilic; cephalic; medial forearm; medial cubital; axillary; deep dorsal; internal pudendal; superficial dorsal; and jugular. In these embodiments, the first vascular location can comprise an artery.

In some embodiments, the method is further constructed and arranged to treat a patient disease or disorder selected from the group consisting of: chronic obstructive pulmonary disease, congestive heart failure, lung fibrosis, adult respiratory distress syndrome; lymphangioleiomytosis; arterial hypertension; pulmonary hypertension; sleep apnea such as sleep apnea due to hypoxemia or hypertension; and combinations thereof.

In some embodiments, the method further comprises dilating the flow pathway, such as a dilation performed with a balloon. The dilation can be performed at a diameter between 1.0 mm and 7.0 mm, such as between 1.0 mm and 6.0 mm, between 1.5 mm and 4.5 mm or between 2.5 mm and 4.0 mm.

In some embodiments, the method further comprises performing a flow pathway assessment procedure, such as an assessment procedure comprising performing an anatomical measurement. The anatomical measurement can comprise a measurement selected from the group consisting of: a flow pathway diameter measurement; a flow pathway length measurement; a measurement of the distance between an artery and vein comprising the flow pathway; a measurement of the distance between the flow pathway and a vessel sidebranch; and combinations thereof. The flow pathway assessment procedure can comprise performing an assessment of at least one of flow in the flow pathway or flow proximate the flow pathway. The assessment can comprise a flow assessment selected from the group consisting of: flow through the flow pathway; flow in a vessel segment proximate the flow pathway; flow measured using Doppler Ultrasound; flow measured using angiographic techniques; and combinations thereof. The flow pathway assessment procedure can comprise an assessment of a patient physiologic condition, such as a patient physiologic condition comprising a condition selected from the group consisting of: an impotence assessment; cardiac output; blood pressure such as systolic and/or diastolic blood pressure; respiration; a blood gas parameter; blood flow such as blood flow through a vein or artery within and/or proximate the penis; vascular resistance; pulmonary resistance; an average clotting time assessment; serum creatinine level assessment; and combinations thereof.

In some embodiments, the method further comprises placing an implant in the flow pathway, such as an anastomotic clip. The implant can comprise an implant selected from the group consisting of: suture; staple; adhesive; and combinations thereof. At least a portion of the implant can comprise biodegradable material.

In some embodiments, the method further comprises modifying the flow pathway. The flow pathway modification can comprise dilating at least a portion of the flow pathway. The method can further comprise placing an anastomotic clip in the flow pathway and the flow pathway modification can be performed after placement of the anastomotic clip. The flow pathway modification can be performed at least one week after the creating of the flow pathway. The flow pathway modification can comprise modifying a flow parameter selected from the group consisting of: flow pathway cross sectional diameter; flow pathway average cross sectional diameter; flow pathway flow rate; flow pathway average flow rate; diastolic pressure after flow pathway creation; diastolic pressure change after flow pathway creation (e.g. as compared to diastolic pressure prior to flow pathway creation); systolic pressure after flow pathway creation; systolic pressure change after flow pathway creation (e.g. as compared to systolic pressure prior to flow pathway creation); ratio of diastolic to systolic pressure after flow pathway creation; difference between diastolic pressure and systolic pressure after flow pathway creation; and combinations thereof. The flow pathway modification can comprise a flow modification procedure selected from the group consisting of: increasing flow through the flow pathway; decreasing flow through the flow pathway; increasing the diameter of at least a segment of the flow pathway; decreasing the diameter of at least a segment of the flow pathway; removing tissue proximate the flow pathway; blocking a sidebranch proximate the flow pathway; and combinations thereof.

In some embodiments, the method further comprises creating a second flow pathway between a third vascular location and a fourth vascular location. The first vascular location can comprise an artery and the third vascular location can comprise the same artery. The second vascular location can comprise a vein and the fourth vascular location can comprise the same vein. The first vascular location can comprise an iliac vein in the right leg of the patient and the third vascular location can comprise an iliac vein in the left leg of the patient. The first vascular location can comprise an iliac vein and the third vascular location can comprises a penile vessel. The second flow pathway can comprise a fistula. The second flow pathway can be created at least twenty four hours after the creation of the first flow pathway.

In some embodiments, the method further comprises placing a flow restricting element within, on and/or proximate the first and/or second vascular location. The flow restricting element can be constructed and arranged to at least partially occlude the first and/or second vascular location. The flow restricting element can be constructed and arranged to fully occlude the first and/or second vascular location. The flow restricting element can be placed in a vein at a location downstream from the flow pathway. The flow restricting element can comprise an element selected from the group consisting of: plug such as a self-expanding vascular plug placed within a blood vessel; a stent with a covered portion such as a vascular stent including one or more covered end portions; a clip such as a vascular clip placed around a blood vessel to limit flow; an implantable clamp; an expandable assembly placed proximate a blood vessel to limit flow; a vessel sealant or occluding agent; and combinations thereof. The flow restricting element can comprise a material selected from the group consisting of: a shape memory material such as nickel titanium alloy; stainless steel; a polymer; a biodegradable material such as magnesium or a bioabsorbable polymer; and combinations thereof.

In some embodiments, the method further comprises delivering energy proximate to the first or second vascular location to at least partially occlude the respective first or second vascular location. The delivered energy can comprise radiofrequency energy.

According to another aspect of the present inventive concepts, a system for treating erectile dysfunction in a patient comprises a needle delivery device and a flow creation device. The needle delivery device is constructed and arranged to place a vessel-to-vessel guidewire from a starting vessel to a target vessel. The flow creation device is constructed and arranged to be advanced over the vessel-to-vessel guidewire and to create a flow pathway between the starting vessel and the target vessel. The system is constructed and arranged to treat erectile dysfunction.

In some embodiments, the system is further constructed and arranged to restrict venous outflow from the penis.

In some embodiments, the system is further constructed and arranged to cause an increase in penile blood pressure, such as penile venous blood pressure.

In some embodiments, the needle delivery device comprises an advanceable needle, such as a needle with a guidewire channel.

In some embodiments, the needle delivery device comprises a needle with a gauge between 20 and 24, such as a needle with a gauge between 22 and 23.

In some embodiments, the needle delivery device comprises a curved needle. The curved needle can comprise a marker indicating the direction of curvature of the curved needle, such as a marker selected from the group consisting of: flat surface, visible marker, line, textured surface, and combinations thereof. The needle delivery device can further comprise a sheath constructed and arranged to slidingly receive the curved needle. The needle can comprise a proximal end and a hub positioned on said proximal end. The hub can be constructed and arranged to be advanced to advance the curved needle out of the sheath.

In some embodiments, the needle delivery device comprises a needle comprising a shaped memory alloy, such as nickel titanium alloy. The needle can comprise an 18 gauge to 24 gauge needle, such as a 22 gauge to 23 gauge needle.

In some embodiments, the system further comprises a vessel-to-vessel guidewire constructed and arranged to be placed from the starting vessel to the target vessel by the needle delivery device. The vessel-to-vessel guidewire can comprise a wire with an outer diameter approximating 0.018″. The vessel-to-vessel guidewire can comprise a wire with an outer diameter between 0.010″ and 0.018″, such as a wire with an outer diameter between 0.010″ and 0.014″. The vessel-to-vessel guidewire can comprise a marker, such as a marker constructed and arranged to be positioned to indicate the fistula location. The vessel-to-vessel guidewire can comprise a distal portion and a mid portion and the mid portion can comprise a construction different than the construction of the distal portion. The mid portion can comprise a stiffness greater than the stiffness of the distal portion.

In some embodiments, the flow creation device comprises a balloon catheter constructed and arranged to dilate tissue positioned between the first vascular location and the second vascular location.

In some embodiments, the flow creation device comprises an energy delivery device constructed and arranged to deliver energy to tissue positioned between the first vascular location and the second vascular location.

In some embodiments, the flow creation device comprises a clip deployment catheter comprising an anastomotic clip. The clip deployment catheter can comprise a length ranging from 10 inches to 18 inches. The clip deployment catheter can comprise a diameter ranging from 7 French (Fr) to 9 Fr. The clip deployment catheter can comprise a handle and the handle can comprise a control constructed and arranged to deploy the anastomotic clip. The control can comprise a button. The handle can comprise a safety position for the control, such as when the handle comprises a longitudinal axis and the control is constructed and arranged to be moved relatively perpendicular to said longitudinal axis to transition from the safety position to a first ready to deploy position. The clip can comprise at least two distal arms, and the handle can be constructed and arranged to allow an operator to move the control from a first ready to deploy position to a first deployed position, such that the movement causes the at least two distal arms to be deployed. The handle can comprise a longitudinal axis and the control can be moved relatively parallel to the longitudinal axis to transition from the first ready to deploy position to the first deployed position. The handle can be constructed and arranged to allow an operator to move the control from the first deployed position to a second ready to deploy position. The control can be constructed and arranged to be moved relatively perpendicular to the longitudinal axis to transition from the first deployed position to the second ready to deploy position. The clip can comprise at least two proximal arms, and the handle can be constructed and arranged to allow an operator to move the control from the second ready to deploy position to a second deployed position, and the movement can cause the at least two proximal arms to be deployed. The control can be constructed and arranged to be moved relatively parallel to said longitudinal axis to transition from the second ready to deploy position to the second deployed position. The clip deployment catheter can comprise an outer sheath and the control can be constructed and arranged to be moved from a first position to a second position, to cause movement of the outer sheath. The clip deployment catheter can be constructed and arranged such that movement of the control to the second position causes a tactile feedback event to occur. The clip can comprise multiple deployable arms, and the clip deployment catheter can be constructed and arranged such that movement of the control to the second position causes at least one arm to be deployed. At least one of the clip deployment catheter or the clip can comprise at least one marker constructed and arranged to rotationally position the clip. The marker can be constructed and arranged to be oriented toward the target vessel prior to deployment of the clip. The marker can be constructed and arranged to be oriented based on a patient image, such as a real-time fluoroscopy image. The clip can comprise a swing arm for deployment in the target vessel and the marker can be positioned in alignment with the swing arm. The marker can be positioned on the clip. The clip deployment catheter can comprise a distal portion and the distal portion can comprise the clip and the marker. The marker can be positioned proximate the clip. The clip deployment catheter can comprise a proximal portion and said proximal portion comprises the marker. The clip deployment catheter can comprise a handle and the marker can be positioned on the handle. At least one of the clip deployment catheter or the clip can comprise at least one marker constructed and arranged to longitudinally position the clip at the fistula location. The marker can indicate the distal end of the clip and/or the proximal end of the clip. The clip can comprise multiple deployable arms, and the clip deployment catheter can be constructed and arranged to deploy at least one of said deployable arms and subsequently recapture said one of the deployable arms. The clip deployment catheter can be constructed and arranged to be rotated and simultaneously deployed from the starting vessel to the target vessel over the vessel-to-vessel guidewire. The clip deployment catheter can comprise a projection constructed and arranged to mechanically engage the clip. The projection can comprise a pin. The clip deployment catheter can further comprise a second projection constructed and arranged to mechanically engage the clip. The clip can comprise an expanded diameter ranging between 1.0 mm and 6.0 mm, such as an expanded diameter between 1.5 mm and 4.5 mm, or between 2.5 mm and 4.0 mm.

In some embodiments, the system further comprises a flow pathway maintaining implant. The flow pathway maintaining implant can comprise an anastomotic clip, such as a clip comprising a plurality of distal arms and a plurality of proximal arms. The distal arms can be constructed and arranged to be independently deployable from the proximal arms. The clip can comprise four deployable distal arms. The clip can comprise four deployable proximal arms. The clip can comprise nickel titanium alloy. The clip can comprise multiple deployable arms and at least two arms can each comprise a marker, such as two radiopaque markers. The flow pathway maintaining implant can comprise suture. The flow pathway maintaining implant can comprise one or more staples. The flow pathway maintaining implant can comprise adhesive. The flow pathway maintaining implant can comprise biodegradable material.

In some embodiments, the system further comprises a venous system introducer. The venous system introducer can be constructed and arranged to access the starting vessel. The venous system introducer can comprise a 10 Fr to 13 Fr introducer. The venous system introducer can comprise a beveled distal tip. The beveled distal tip can comprise an angle between 20° and 50°, such as an angle of approximately 30°. The venous system introducer can comprise a marker proximate the beveled distal tip, such as a radiopaque marker. The venous system introducer can comprise a proximal portion comprising a marker, and the marker can be aligned with the beveled distal tip. The venous system introducer can comprise a distal portion and an expandable element mounted to the distal portion. The expandable element can comprise a balloon. The expandable element can be constructed and arranged to prevent inadvertent advancement of the introducer into the target vessel. The venous system introducer can be constructed and arranged to stabilize the starting vessel.

In some embodiments, the system further comprises an arterial system introducer. The arterial system introducer can be constructed and arranged to access the target vessel. The arterial system introducer can comprise approximately a 4 Fr introducer.

In some embodiments, the system further comprises a target wire constructed and arranged for positioning in the target vessel. The target wire can comprise a helical distal portion. The target wire can comprise a radiopaque distal portion.

In some embodiments, the system further comprises a flow pathway modifying device, such as a flow pathway modifying device comprising an expandable element. The expandable element can be constructed and arranged to expand to a diameter between 1.0 mm and 7.0 mm, such as to a diameter between 1.0 mm and 6.0 mm, between 1.5 mm and 4.5 mm or between 2.5 mm and 4.0 mm. The expandable element can comprise a balloon, an expandable cage and/or radially deployable arms. The flow modifying device can comprise a device selected from the group consisting of: an over the wire device constructed and arranged to be delivered over a vessel-to-vessel guidewire as described herein; an expanding scaffold constructed and arranged to increase or otherwise modify flow pathway geometry such as an expandable balloon; an energy delivery catheter such as a catheter constructed and arranged to deliver energy to tissue proximate a flow pathway; an agent delivery catheter such as a catheter constructed and arranged to deliver an agent such as a pharmaceutical agent or an adhesive such as fibrin glue; and combinations thereof.

In some embodiments, the system further comprises a patient imaging apparatus, such as a fluoroscope or an ultrasound imager.

In some embodiments, the system further comprises a flow restricting element constructed and arranged to at least partially occlude at least one of the starting vessel or the target vessel. The flow restricting element can be constructed and arranged to fully occlude at least one of the starting vessel or the target vessel. The flow restricting element can be constructed and arranged to be placed in a vein downstream from the flow pathway. The flow restricting element can comprise an element selected from the group consisting of: a plug such as a self-expanding vascular plug placed within a blood vessel; a stent with a covered portion such as a vascular stent including one or more covered end portions; a clip such as a vascular clip placed around a blood vessel to limit flow; an implantable clamp; an expandable assembly placed proximate a blood vessel to limit flow; a vessel sealant or occluding agent; and combinations thereof. The flow restricting element can comprise a material selected from the group consisting of: a shape memory material such as nickel titanium alloy; stainless steel; a polymer; a biodegradable material such as magnesium or a bioabsorbable polymer; and combinations thereof.

In some embodiments, the system further comprises an energy delivery device configured to deliver energy to the starting vessel or the target vessel to at least partially occlude the respective starting vessel or the target vessel. The delivered energy can comprise radiofrequency energy.

In some embodiments, the system is further constructed and arranged to treat a patient disease or disorder selected from the group consisting of: chronic obstructive pulmonary disease, congestive heart failure, lung fibrosis, adult respiratory distress syndrome; lymphangioleiomytosis; arterial hypertension; pulmonary hypertension; sleep apnea such as sleep apnea due to hypoxemia or hypertension; and combinations thereof.

According to another aspect of the inventive concepts, a system for creating a fistula between a starting vessel and a target vessel at a fistula location in a patient comprises a vascular introducer, a needle delivery device; a vessel-to-vessel guidewire, an anastomotic clip and a clip deployment catheter. The vessel-to-vessel guidewire is constructed and arranged to be placed from the starting vessel to the target vessel by the needle delivery device. The clip deployment catheter is constructed and arranged to deploy the anastomotic clip. The system is constructed and arranged to treat erectile dysfunction.

In some embodiments, the system is further constructed and arranged to treat a patient disease or disorder selected from the group consisting of: erectile dysfunction; chronic obstructive pulmonary disease, congestive heart failure, lung fibrosis, adult respiratory distress syndrome; lymphangioleiomytosis; arterial hypertension; pulmonary hypertension; sleep apnea such as sleep apnea due to hypoxemia or hypertension; and combinations thereof.

The technology described herein, along with the attributes and attendant advantages thereof, will best be appreciated and understood in view of the following detailed description taken in conjunction with the accompanying drawings in which representative embodiments are described by way of example

### BRIEF DESCRIPTION OF THE DRAWINGS

The accompanying drawings, which are incorporated in and constitute a part of this specification, illustrate various embodiments of the present inventive concepts, and, together with the description, serve to explain the principles of the invention. In the drawings:

FIG. 1 is a flow chart of a method for treating a patient by creating a flow pathway between a first vascular location and a second vascular location, consistent with the present inventive concepts.

FIG. 2 is a schematic view of a system for creating a flow pathway in a patient, consistent with the present inventive concepts.

FIGS. 3A through 3D are a set of steps for implanting an anastomotic clip, consistent with the present inventive concepts.

FIG. 4 is a flow chart of a method for treating a patient with a flow pathway, consistent with the present inventive concepts.

FIG. 5 is a schematic of an angiographic view of a patient's vein and artery prior to advancement of a needle into the artery, consistent with the present inventive concepts.

FIGS. 5A, 5B and 5C are anatomical views of three different needle trajectory paths, consistent with the present inventive concepts.

FIG. 6 is a perspective view of an anastomotic clip, consistent with the present inventive concepts.

FIG. 7 is a sectional view of a flow pathway created between a first vascular location and a second vascular location, with a flow restricting element implanted in the first vascular location, consistent with the present inventive concepts.

### DETAILED DESCRIPTION OF THE DRAWINGS

Reference will now be made in detail to the present embodiments of the inventive concepts, examples of which are illustrated in the accompanying drawings. Wherever practical, the same reference numbers will be used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

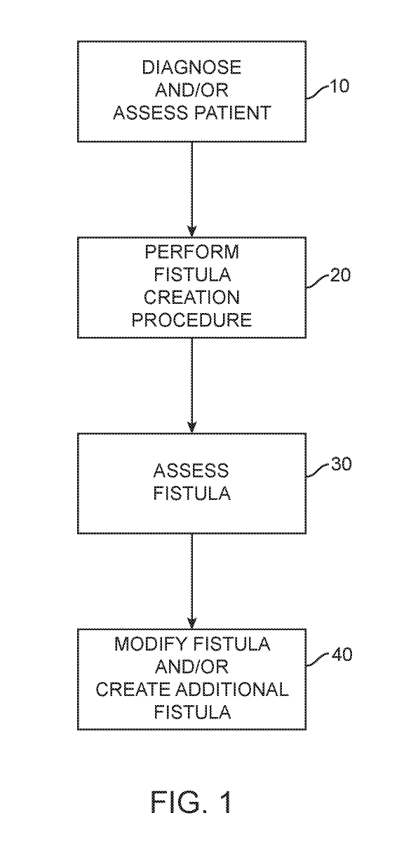

Referring now to FIG. 1, a flow chart for selecting and treating a patient by creating a fistula or other flow pathway between a first vascular location in the patient's arterial system and a second vascular location in the patient's venous system is illustrated, consistent with the present inventive concepts. In STEP 10, a patient assessment is performed, such as to diagnose the patient and determine if a fistula should be created in the patient. A patient can be selected based on a disease or disorder which is diagnosed in STEP 10 or previously. In some embodiments, a patient diagnosed with or otherwise exhibiting erectile dysfunction is selected to receive a fistula. Alternatively or additionally, a patient selected to receive a fistula can have a disease or disorder selected from the group consisting of: chronic obstructive pulmonary disease (COPD), congestive heart failure, lung fibrosis, adult respiratory distress syndrome; lymphangioleiomytosis; arterial hypertension; pulmonary hypertension; sleep apnea such as sleep apnea due to hypoxemia or hypertension; and combinations of these.

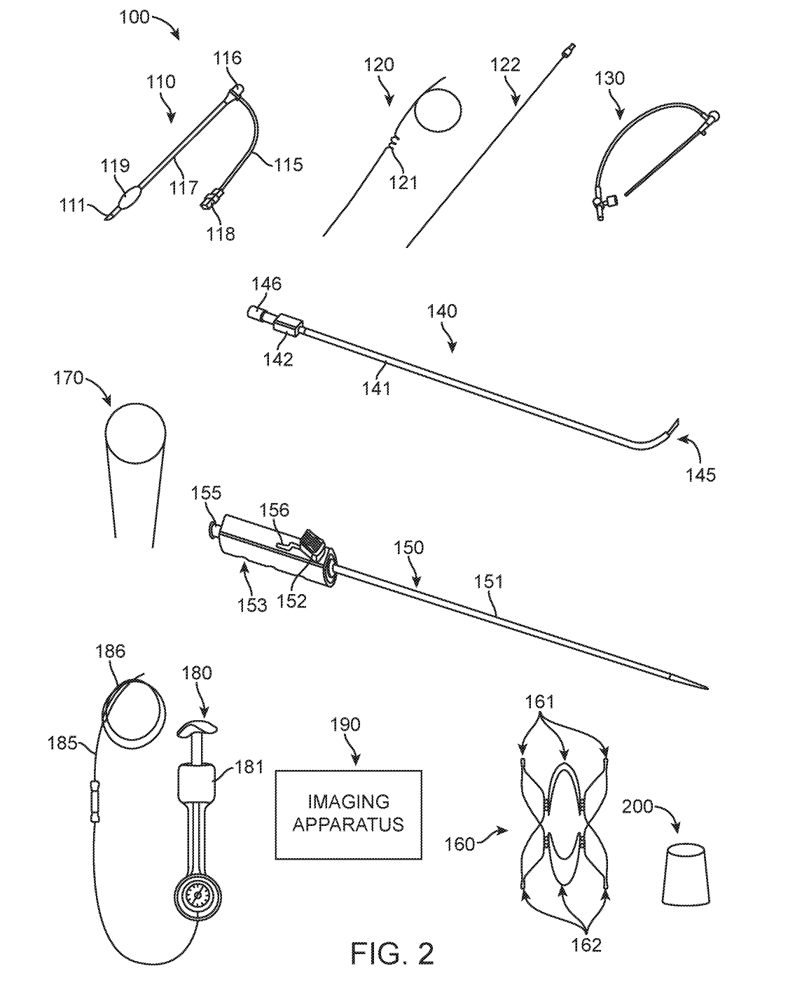

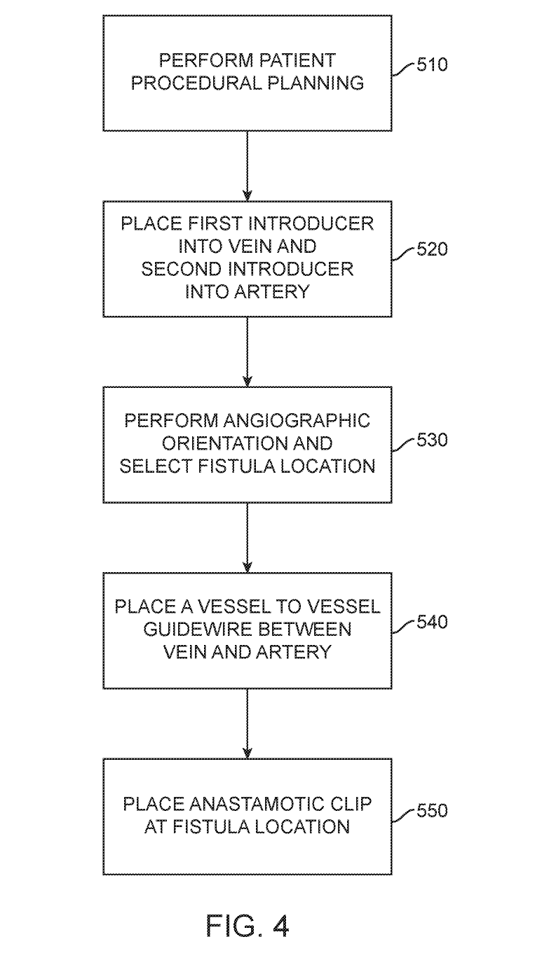

In STEP 20, a fistula creation procedure is performed on the patient. In some embodiments, the fistula creation procedure is performed as described in reference to FIG. 4 herebelow. In some embodiments, the fistula creation procedure is performed using a system of devices and components similar to system 100 of FIG. 2 described herebelow. The fistula is created between a first vascular location in the arterial system, such as an artery, and a second vascular location in the venous system, such as a vein. The fistula creation procedure can include the placement of a vessel-to-vessel guidewire between a starting vessel such as a vein, and a target vessel such as an artery. In these embodiments, the fistula can be created using one or more fistula creation devices that are advanced over the vessel-to-vessel guidewire. An anastomotic clip or other implant can be placed into the fistula via a clip placement device advanced over the vessel-to-vessel guidewire. Alternatively, a fistula can be created without an anastomotic clip, such as through the use of energy (e.g. radiofrequency energy), suture or staple (e.g. via an over-the-wire suture or staple delivery device), and/or a tissue treatment such as an adhesive (e.g. fibrin glue) coating of the tissue surrounding or otherwise proximate the fistula. One or more fistula treatment or modification procedures can be performed using fistula treatment or modification devices advanced over the vessel-to-vessel guidewire, such as a fistula modification performed in STEP 40 herebelow.

In some embodiments, a fistula or other flow pathway is created between an artery and a vein at a location positioned to treat erectile dysfunction, such as at a location within and/or proximate the vasculature of the penis. In some embodiments, the fistula is positioned such that it is constructed and arranged to increase blood flow to the penis and/or to increase penile blood pressure (e.g. at least during arousal) such as by restricting the venous outflow from the penis (e.g. to decrease venous drainage or otherwise decrease venous outflow), by increasing local venous pressure, and/or by converting penile veins into arteries via AV anastomosis combined with down stream occlusion. Numerous locations for the fistula or other flow pathway can be selected, such as a fistula located between an artery and vein as described in reference to FIG. 4 herebelow. In some embodiments, multiple flow pathways are created, such as to reduce the cross-sectional area of at least one flow pathway created. Alternatively or additionally, arterial blood can be diverted to the venous system by way of a flow pathway comprising a bypass graft, such as is described in applicant's U.S. Pat. No. 8,016,782, entitled “Methods for Providing Oxygenated Blood to Venous Circulation”, filed Jun. 13, 2005, the contents of which are incorporated by reference herein in its entirety. In some embodiments, one or more flow pathways are created to reduce hypertension, such as to cause a reduction in hypertension combined with an increase in local penile venous pressure.

During the fistula creation procedure and/or in a subsequent fistula modification procedure, a fistula dilation procedure can be performed. In some embodiments, an anastomotic clip is placed in the fistula and a balloon catheter is used to dilate the fistula and anastomotic clip simultaneously. In some embodiments, the balloon comprises a diameter between 1.0 mm to 7.0 mm, such as a diameter between 1.0 mm and 6.0 mm, 1.5 mm and 4.5 mm, or 2.5 mm and 4.0 mm.

In STEP 30, a fistula assessment procedure can be performed. STEP 30 can be performed in the same clinical procedure as STEP 20, and/or in a subsequent clinical procedure such as a procedure at least twenty-four hours after completion of STEP 20, or at least 1 week, at least 1 month, and/or at least 6 months after completion of STEP 20. In some embodiments, the assessment performed in STEP 30 includes one or more anatomical measurements, such as a measurement selected from the group consisting of: a fistula diameter measurement; a fistula length measurement; a measurement of the distance between the artery and vein comprising the fistula; a measurement of the distance between the fistula and a vessel sidebranch; and combinations of these. In some embodiments, the assessment performed in STEP 30 comprises an assessment of flow, such as a flow assessment selected from the group consisting of: flow through the fistula; flow in a vessel segment proximate the fistula; flow measured using Doppler Ultrasound; flow measured using angiographic techniques; and combinations of these. In some embodiments, the assessment performed in STEP 30 comprises an assessment of a patient physiologic condition, such as an assessment of a physiologic condition selected from the group consisting of: an impotence assessment; cardiac output; blood pressure such as systolic and/or diastolic blood pressure; respiration; a blood gas parameter; blood flow such as blood flow through a vein or artery within and/or proximate the penis; vascular resistance; pulmonary resistance; an average clotting time assessment; serum creatinine level assessment; a condition assessed during an arteriogram, venogram and/or vascular ultrasound; and combinations of these.

In STEP 40, one or more fistula parameters can be modified. STEP 40 can be performed in the same clinical procedure as STEP 20, and/or in a subsequent clinical procedure such as a procedure at least twenty-four hours after completion of STEP 20, or at least 1 week, at least 1 month, and/or at least 6 months after completion of STEP 20. In some embodiments, STEP 30 and STEP 40 are performed in the same clinical procedure (e.g. both in the same clinical procedure as STEP 20 or both in a subsequent clinical procedure). In some embodiments, one or more patient or fistula parameters to be modified are selected from the group consisting of: fistula cross sectional diameter; fistula average cross sectional diameter; fistula flow rate; fistula average flow rate; diastolic pressure after fistula creation; diastolic pressure change after fistula creation (e.g. as compared to diastolic pressure prior to fistula creation); systolic pressure after fistula creation; systolic pressure change after fistula creation (e.g. as compared to systolic pressure prior to fistula creation); ratio of diastolic to systolic pressure after fistula creation; difference between diastolic pressure and systolic pressure after fistula creation; and combinations of these.

Fistula modification procedures can include but are not limited to: increasing flow through the fistula; decreasing flow through the fistula; increasing the diameter of at least a segment of the fistula; decreasing the diameter of at least a segment of the fistula; removing tissue proximate the fistula; blocking a sidebranch proximate the fistula; and combinations of these. A fistula modifying device can include one or more devices selected from the group consisting of: an over the wire device constructed and arranged to be delivered over a vessel-to-vessel guidewire as described herein; an expanding scaffold configured to increase or otherwise modify fistula geometry such as an expandable balloon; an energy delivery catheter such as a catheter configured to deliver energy to tissue proximate a fistula; an agent delivery catheter such as a catheter configured to deliver an agent such as a pharmaceutical agent or an adhesive such as fibrin glue; and combinations of these.

In some embodiments, a second fistula is created, such as using the techniques of STEP 20 described hereabove. The second fistula can be created in the same clinical procedure as STEP 20 (in which the first fistula is created), or in a subsequent clinical procedure such as a procedure performed at least twenty-four hours after completion of STEP 20, or at least 1 week, at least 1 month, and/or at least 6 months after completion of STEP 20. A second fistula can be created due to inadequate therapy provided by the first fistula, and/or if the first fistula has insufficient flow (e.g. becomes non-patent). A second or third fistula can be created, such as to allow smaller cross-sectional areas for one or more of the created fistulas. Smaller cross-sectional areas can be created to reduce turbulence and/or reduce the tendency for venous stenosis formation. A second fistula can be created due to formation of a vascular (e.g. venous) stenosis proximate the first fistula. In these embodiments, the first fistula can be reversed (e.g. closed), such as through the placement of a covered stent graft in the vein or artery that covers the fistula, or other fistula-occlusive procedure. In some embodiments, a first fistula is created between an iliac vein and artery in the right leg of the patient, and a second fistula is created between an iliac vein and artery in the left leg of the patient. In other embodiments, a first fistula is created between an iliac vein and artery in one leg of the patient (e.g. the right leg or the left leg), and a second fistula is created between two penile vessels.

In some embodiments, a flow restricting element is placed within, on and/or proximate an artery or the vein of the patient, to at least partially occlude or fully occlude flow in the respective artery or vein. In some embodiments, the flow restricting element can be placed within, on and/or proximate a vein, at a location downstream from the flow pathway. In these embodiments, the flow restricting element can be constructed and arranged to increase flow in a retrograde direction through the vein and into the penis and/or to increase blood pressure in an artery and/or vein of the penis. Examples of a flow restricting element are described in FIG. 2 herebelow. In some embodiments, the method can further include delivering energy to the artery or vein to at least partially occlude or fully occlude the respective artery or vein, such as without an implant. For example, the energy can comprise radiofrequency energy delivered to modify tissue (e.g. blood vessel tissue) to restrict flow in a blood vessel.

The method of FIG. 1 can be performed using real-time imaging, such as real-time imaging provided by a fluoroscope and/or an ultrasound imaging device.

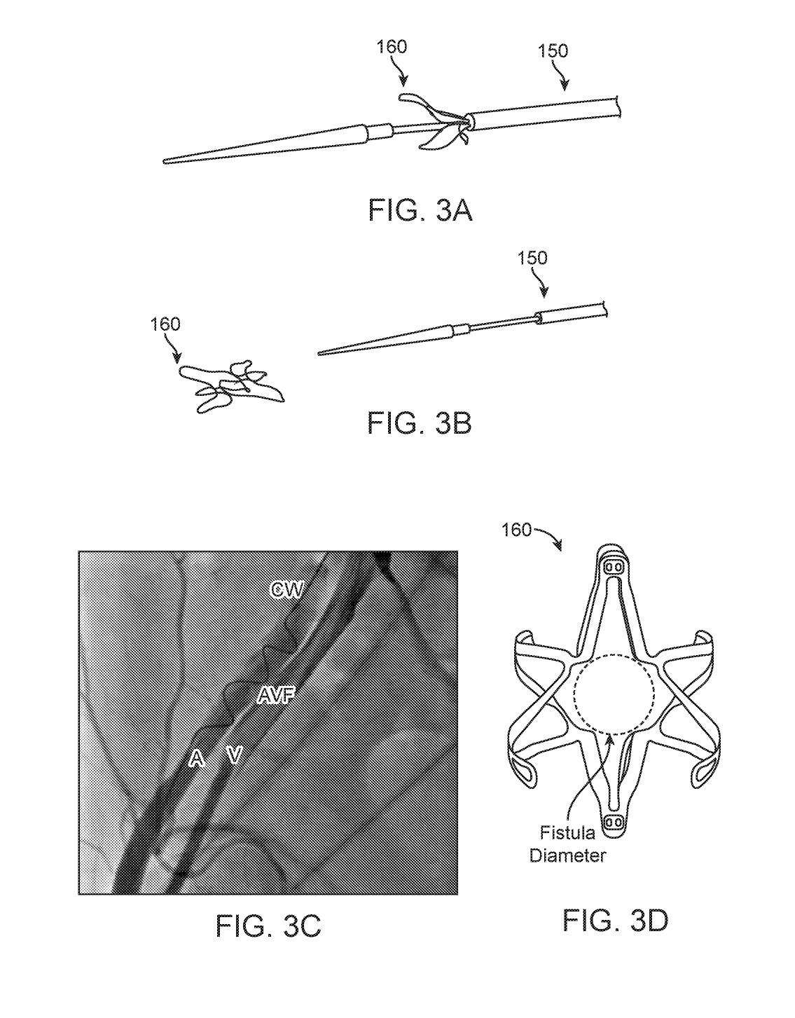

In an exemplary procedure illustrated in FIGS. 3A-D, an anastomotic clip can be deployed to create an iliac arteriovenous fistula. Vascular venous and arterial access can be obtained using standard interventional techniques (e.g. through a femoral vein and femoral artery). FIGS. 3A and 3B illustrate an anastomotic clip delivery device that can be used, including an anastomotic clip that can be implanted. In some embodiments, the anastomotic clip delivery device comprises clip deployment catheter 150, and the anastomotic clip comprises clip 160, each of FIG. 2 herebelow. In FIG. 3C, an angiogram of the fistula location AVF is illustrated, including artery A and vein V, prior to fistula creation. A radiopaque vessel targeting wire CW, such as wire 121 of FIG. 2, can be inserted within artery A to provide a radiographic outline of the artery. A venogram can be performed to outline the vein and confirm proximity of the artery and vein at the target crossing location AVF for the creation of the arteriovenous fistula. A crossing needle device (e.g. a 22 gauge or 23 gauge crossing needle device), such as deployment device 140 of FIG. 2 herebelow, can be placed into vein V, such as a placement over a guidewire and through an introducer device (e.g. introducer 110 of FIG. 2). The crossing needle of the device can be advanced through the wall of vein V into artery A, and a guidewire can be advanced through a lumen of the crossing needle and into artery A. The crossing needle can be subsequently removed and the anastomotic clip delivery system can be tracked across the puncture site. The anastomotic clip can then be deployed so that the expanded arms of the anastomotic clip attach to the inner walls of artery A and vein V, and the retention arms maintain the anastomotic clip in the proper position (deployed position shown in FIG. 3D). After removal of the delivery system, a balloon catheter (e.g. a 2.5-4.5 mm balloon catheter) can be inserted into the center of the anastomotic clip and inflated to expand the anastomotic clip to a target diameter as described herein. The balloon can then be deflated and removed. An angiogram can confirm the patency of the fistula.

Referring now to FIG. 2, a system for creating a fistula or other flow pathway between a first location in a patient's arterial system of a patient (e.g. an artery), and a second location in the patient's venous system (e.g. a vein), is illustrated. System 100 comprises a vascular introducer, first introducer 110, configured to be placed into the patient to provide access to a starting vessel. System 100 comprises another vascular introducer, second introducer 130, configured to provide access to a target vessel. In some embodiments, the starting vessel is a vein, and the target vessel is an artery. In other embodiments, the starting vessel is an artery and the target vessel is a vein. System 100 can include target wire 120 which comprises helical section 121 and is configured to be placed through the second introducer 130 and into the target vessel. Target wire 120 can be placed through an elongate tube, catheter 122. System 100 can comprise needle deployment device 140 which is configured to deploy crossing needle 141 (shown in an advanced position in FIG. 2), from the starting vessel and into the target vessel. Crossing needle 141 can comprise a needle with a gauge between 18 and 24, such as a needle with a gauge between 22 and 23. Crossing needle 145 includes a guidewire channel configured to allow advancement of one or more guidewires through needle 145. System 100 can include a vessel-to-vessel guidewire 170, which can be placed from the starting vessel to the target vessel via needle deployment device 140. Guidewire 170 can comprise a guidewire with an outer diameter between 0.010″ and 0.018″, such as a guidewire with a diameter between 0.010″ and 0.014″. System 100 can also include clip deployment catheter 150, which is configured to deploy anastomotic clip 160. System 100 can include a fistula modifying device, such as dilation device 180 including balloon catheter 185 and standard angioplasty balloon indeflator 181. System 100 can further comprise imaging apparatus 190, typically a fluoroscope and/or ultrasound imaging device used to image one or more device or components of system 100, as well as the patient's anatomy, during the creation of an arteriovenous fistula.

First introducer 110 is configured to be placed into the patient to provide access to a starting vessel (e.g. a vein of a patient). In some embodiments, introducer 110 comprises an 11 Fr vascular introducer. First introducer 110 can comprise beveled tip 111 with an angle ranging from 20° to 500, such as at an angle of approximately 30°. Additionally, system 100 can include a kit comprising an additional introducer having a second angle providing the clinician or other user (hereinafter “clinician”) with more options as may be appropriate for a particular patient's anatomical geometry. In some embodiments, beveled tip 111 comprises a marker, for example, a radiopaque or other visualizable marker, such that the luminal wall of the starting vessel can be imaged (e.g. when tip 111 is pressed against the vessel wall). The proximal portion of introducer 110 can comprise a contour or marker, such as to be correlated with or otherwise indicate the alignment of the bevel of tip 111.

Introducer 110 comprises shaft 117, which includes at least one thru lumen. Introducer 110 also comprises port 116, typically a hemostasis valve, which is fluidly connected to the lumen of shaft 117. A second port 118, typically a luer connector, is connected to tubing 115 which in turn is connected to port 116. Introducer 110 can further comprise a dilator, not shown but typically a 10 to 13 Fr dilator used to introduce and/or pre-dilate tissue receiving introducer 110. Introducer 110 can further comprise a radially expandable element, such as expandable element 119, such as a balloon or expandable cage located on its distal portion. In some embodiments, expandable element 119 can be configured to prevent advancement of introducer 110 into the target vessel. In yet another embodiment, expandable element 119 can be configured to stabilize the starting vessel during insertion of introducer 110 or another device or component of system 100.

System 100 can comprise second introducer 130 which is configured to provide access to a target vessel, such as an artery of the patient when the starting vessel is a vein. In some embodiments, second introducer 130 comprises a 4 Fr vascular introducer. System 100 comprises target wire 120 configured to be placed through second introducer 130 and into the target vessel. Target wire 120 can comprise helical section 121 configured to be deployed at the site where the fistula is to be created. Helical section 121 can be configured to provide structure and support to the site during a procedure. Additionally, target wire 120 can serve as a visual reference during insertion of vessel-to-vessel guidewire 170, as described herebelow.

System 100 can comprise needle deployment device 140. Needle deployment device 140 comprises shaft 141, which slidingly receives advanceable crossing needle 145, shown in an advanced state. Shaft 141 comprises shaft hub 142 mounted to its proximal end. Shaft 141 can comprise a curved distal portion as shown. Crossing needle 145 comprises needle hub 146 mounted to its distal end. Movement of needle hub 146 relative to shaft hub 142 causes crossing needle 145 to advance and retract within shaft 141. Needle hub 146 is fully advanced toward shaft hub 142 in the configuration of FIG. 2, such that the tip and distal portion of crossing needle 145 is fully advanced out of the distal end of shaft 141.

Crossing needle 145 can comprise a 20 to 24 gauge needle, such as a 22 gauge needle. In some embodiments, the crossing needle comprises a curved distal portion (as shown). The curved distal portions of shaft 141 and/or needle 145 can be aimed at the center of the target vessel prior to insertion into the target vessel. The radius of curvature can be reduced if the clinician has difficulty in aiming the needle tip at the center of the target vessel prior to insertion. Conversely, the radius of curvature can be increased to sufficiently aim the needle tip at the center of the target vessel. Additionally, the crossing needle 145 can comprise a marker, not shown but indicating the direction of curvature. Examples of markers include, but are not limited to: a flat surface, a textured surface; a visualizable marker such as a radiopaque marker, a magnetic marker, an ultrasonic marker or a visible marker; and combinations of these. In some embodiments, crossing needle can comprise a shaped memory alloy, for example, nickel titanium alloy. In some embodiments, shaft hub 142 and/or needle hub 146 comprise a marker or other visible demarcation (e.g. a flat portion) which correlates to the direction of curvature of shaft 141 and/or crossing needle 145, respectively.

System 100 can comprise a guidewire to be placed from the starting vessel to the target vessel, vessel-to-vessel guidewire 170. Guidewire 170 is configured to be placed via needle deployment device 140. In some embodiments, vessel-to-vessel guidewire 170 comprises a wire with an outer diameter of approximately 0.018″. Vessel-to-vessel guidewire 170 can comprise a marker, not shown but configured to indicate the fistula location. In some embodiments, vessel-to-vessel guidewire 170 comprises a distal portion and a mid portion. Guidewire 170 mid portion can comprise a different construction than the distal portion. For example, the mid portion of guidewire 170 can be stiffer than the distal portion.

System 100 can comprise clip deployment catheter 150 configured to house and deploy anastomotic clip 160. Clip 160 comprises a plurality of distal arms 161 and a plurality of proximal arms 162, which can be deployed simultaneously or independently. Clip 160 comprises at least two distal arms 161 and at least two proximal arms 162 configured to deploy and engage the starting vessel and the target vessel. In some embodiments, clip 160 comprises four deployable distal arms 161 and four deployable proximal arms 162. Clip 160 can comprise a shaped memory alloy, such as nickel titanium alloy. In some embodiments, clip 160 is constructed and arranged as described in applicant's U.S. Pat. No. 7,828,814, entitled “Device and Method for Establishing an Artificial Arterio-Venous Fistula”, filed Apr. 4, 2007, the contents of which are incorporated herein by reference in its entirety. Clip 160 can comprise an expanded diameter, i.e. diameter of clip 160 after deployment, can range between 1.0 mm and 6.0 mm, for example between 1.5 mm and 4.5 mm or 2.5 mm and 4.0 mm.

In some embodiments, clip 160 is biodegradable or includes one or more biodegradable portions (e.g. one or more portions of clip are absorbed or otherwise degrade over time). In some embodiments, clip 160 comprises a biodegradable anastomotic device such as is described in applicant's co-pending U.S. Non-Provisional application Ser. No. 12/752,397, entitled “Device and Method for Establishing an Artificial Arteriovenous Fistula”, filed Apr. 1, 2010, the contents of which are incorporated herein by reference in its entirety.

Clip deployment catheter 150 comprises shaft 151. Mounted to the proximal end of shaft 151 is handle 153. On the proximal end of handle 153 is port 155, which is operably attached to shaft 151, such that a guidewire can travel from the distal end of shaft 151 to port 155, such as guidewire 170 after it has been previously placed between a starting vessel and a target vessel as has been described hereabove. Shaft 151 comprises one or more tubular portions, such as an inner tubular segment that houses clip 160, and an outer tubular segment that covers clip 160 but can be retracted to deploy clip 160, such as is described in applicant's U.S. Pat. No. 8,641,747, entitled “Devices for Arterio-Venous Fistula Creation”, filed Jun. 13, 2005, the contents of which is incorporated herein by reference in its entirety. In some embodiments, clip deployment catheter 150 comprises a length ranging from 10 inches to 18 inches. In some embodiments clip deployment catheter 150 comprises a diameter ranging from 7 Fr to 9 Fr.

Handle 153 further includes control 152 (e.g. a button, slide or lever), where control 152 is operably configured to allow an operator to deploy distal arms 161 and/or proximal arms 162 of clip 160, such as via retraction of an outer tube or sheath portion of shaft 151 that is covering one or more portions of clip 160. In some embodiments, a click or other tactile feedback is provided during retraction of a sheath portion of shaft 151. Control 152 can be moved via a stepped or otherwise segmented slot 156. Distal arms 161 can be deployed via moving control 152 from a “first ready to deploy” position to a “first deployed” position, which can be achieved by moving control 152 relatively parallel to the longitudinal axis of handle 153. The at least two proximal arms 162 can be queued to be deployed via moving control 152 from the first deployed position to a “second ready to deploy” position. The second ready to deploy position can be achieved by moving control 152 in a direction perpendicular to the longitudinal axis of the handle. Subsequently, proximal arms 162 can deployed via moving control 152 from the second ready to be deployed position to a “second deployed” position via a motion parallel to the longitudinal axis of the handle. In this embodiment, control 152 can include a safety position comprising a ready to deploy position which can be transitioned by moving control 152 in a direction that is perpendicular to the axis of handle 153. This control advancement arrangement can prevent inadvertent deployment of distal arms 161 and/or proximal arms 162.

In some embodiments, prior to deployment of one or more arms of clip 160, introducer 110 can be advanced such that tip 111 applies a force to the wall of the starting vessel. Sufficient force can be applied by introducer 110 to enable an operator to “seat” the starting vessel against the target vessel to assist in proper deployment of clip 160.

In some embodiments, catheter 150 can be configured to recapture distal arms 161 and/or proximal arms 162. For example, clip deployment catheter 150 can deploy at least one distal arm 161 and subsequently recapture the at least one distal arm 161.

Clip deployment catheter 150 and/or clip 160 can further comprise at least one marker, not shown but typically a radiopaque and/or ultrasonic marker configured to assist in the rotational positioning of clip 160 at the fistula location. For example, the marker can be oriented toward the target vessel prior to deployment of clip 160. In some embodiments, a marker is included on the distal portion of clip deployment catheter 150. In some embodiments, handle 153 comprises one or more markers that are circumferentially aligned with clip 160 prior to its deployment. In some embodiments, clip deployment catheter 150 and/or clip 160 comprise at least one marker configured to longitudinally position clip 160 at the fistula location. In these embodiments, the marker can indicate the distal and/or proximal end of clip 160.

Clip deployment catheter 150 can further comprise a projection and/or recess, neither shown but configured to mechanically engage clip 160. The project and/or pin can be used to stabilize clip 160 with shaft 151, such as when an outer tubular portion of shaft 151 is advanced or retracted.

System 100 can comprise flow restricting element 200 configured to be placed within, on and/or proximate a blood vessel of the patient to occlude or simply reduce flow in that blood vessel. In some embodiments, flow restricting element 200 is placed within, on and/or proximate the first or second vascular location to at least partially occlude or fully occlude flow within the respective first or second vascular location (e.g. fully or partially restrict flow in an artery or vein at a location proximate the created flow pathway). In some embodiments, flow restricting element 200 can be placed to restrict flow in a vein downstream from the flow pathway, such as to increase flow in a retrograde direction through the vein and into the penis and/or to increase blood pressure in an artery and/or vein of the penis. Flow restricting element 200 can comprise an element selected from the group consisting of: a plug such as a self-expanding vascular plug placed within a blood vessel; a stent with a covered portion such as a vascular stent including one or more covered end portions; a clip such as a vascular clip placed around a blood vessel to limit flow; an implantable clamp; an expandable assembly placed proximate a blood vessel to limit flow; a vessel sealant or occluding agent; and combinations of these. In some embodiments, flow restricting element 200 is constructed and arranged as described in applicant's U.S. Pat. No. 8,226,592, entitled “Method of Treating COPD with Artificial Arterio-Venous Fistula and Flow Mediating Systems”, filed Dec. 15, 2004, the contents of which is incorporated herein by reference in its entirety. Flow restricting element 200 can comprise a material selected from the group consisting of: a shape memory material such as nickel titanium alloy; stainless steel; a polymer; a biodegradable material such as magnesium or a bioabsorbable polymer; and combinations of these. In some embodiments, system 100 can further include an energy delivery element or device, not shown but configured to delivery energy to the first or second vascular location to at least partially occlude or fully occlude the respective first or second vascular location. For example, the energy can comprise radiofrequency energy delivered to modify tissue (e.g. blood vessel tissue) to restrict flow in a blood vessel.

System 100 can comprise dilation device 180 configured to dilate clip 160 and/or the fistula. Dilation device 180 can include balloon catheter 185, such as a standard angioplasty balloon catheter comprising balloon 186. Attached to the proximal end of catheter 185 is indeflator 181, typically a standard balloon indeflator device. Alternatively, balloon 186 can comprise a non-balloon expandable such as an expandable cage or radially deployable arms configured to dilate the fistula. Catheter 185 is configured to track over a vessel-to-vessel guidewire, such as guidewire 170 placed between a vein and an artery, such that balloon 186 is positioned within the fistula (e.g. within clip 160). In some embodiments, dilation device 180 can expand to a diameter between 1.0 mm and 7.0 mm, for example to a diameter between 1.0 mm and 6.0 mm, 1.5 mm and 4.5 mm, or between 2.5 mm and 4.0 mm. In some embodiments, a second dilation device 180 is included, such as a device configured to expand to a different diameter than the first dilation device.

System 100 can include patient imaging apparatus 190. Non-limiting examples of an imaging apparatus include: x-ray; fluoroscope; ultrasound imager; MRI; and combinations of these. The imaging apparatus can allow the clinician to track the movement of all components comprising system 100 as well as view the position of the starting and target vessel relative to each other, as described in detail herein.

Referring now to FIG. 4, a flow chart of a method of creating a fistula between a starting vessel and a target vessel at a fistula location, consistent with the present inventive concepts is illustrated. In Step 510, a procedural planning assessment of a patient is performed. Step 520 comprises placing a first introducer into a starting vessel, e.g. a vein, and placing a second introducer into a target vessel, e.g. an artery. In Step 530, an angiographic orientation is performed and a fistula location is selected. Step 540 comprises placing a vessel-to-vessel guidewire between the vein and the artery. Step 550 comprises placing an anastomotic clip at the fistula location. In some embodiments, system 100 and/or one or more components of system 100 of FIG. 2 are used to perform the method of FIG. 4.

The starting vessel can comprise a vein, and can be selected from the group consisting of: inferior vena cava (IVC); saphenous; femoral; iliac; popliteal; brachial; basilic; cephalic; medial forearm; medial cubital; axillary; deep dorsal; internal pudendal; superficial dorsal; and jugular. The target vessel can comprise an artery, and can be selected from the group consisting of: aorta; axillary; brachial; ulnar; radial; profundal; femoral; iliac; popliteal; internal pudendal; bulbourethral; dorsal; cavernosal; and carotid. In a preferred embodiment, the starting vessel and target vessel comprise an external iliac. In an alternate embodiment, the starting vessel can comprise an artery and the target vessel can comprise a vein.

Step 510, the first step in the illustrated method of the present inventive concepts comprises procedural planning. This step comprises properly orienting the vein and the artery, meaning a clinician becomes familiar with the anatomical orientation of the vein and artery relative to each other. Understanding the orientation of the vessels with respect to one another can be achieved through analysis of one or more images provided by an imaging apparatus (e.g. a fluoroscope) such as imaging apparatus 190 of FIG. 2. In some embodiments, at least one of the vein or artery has a diameter of ranging from 1.0 mm to 7.0 mm proximate the fistula location, for example a diameter between 1.0 mm and 6.0 mm, 1.5 mm and 4.5 mm, or 2.5 mm and 4.0 mm.

In Step 520, the method comprises placing a first introducer into the vein. Preferably, the first introducer comprises an 11 Fr introducer having a beveled tip, such as introducer 110 of FIG. 2 described hereabove. In some instances, the beveled tip of the first introducer can be rotated during insertion into the vein. Rotation of the introducer can be helpful during insertion into the starting vessel due to the tendency of the beveled tip to lift and pull back. Additionally or alternatively, the introducer can be vibrated while it is advanced into the vein. Step 520 can further comprise pre-dilating the vein with a dilator, preferably a 13 Fr dilator, prior to placing the introducer into the vein. Additionally, a second introducer can be placed into the artery. Preferably, the second introducer comprises a 4 Fr introducer, such as second introducer 130 described in FIG. 2 hereabove. The method further comprises placing a target wire into the second introducer and then into the artery such that the distal end of the target wire is positioned five to ten centimeters past the fistula location, and configured to serve as a visual reference to a clinician. The target wire, typically including a helical section, is advanced. The advancement can be combined with retracting the introducer such that the helical section of the wire is deployed at the targeted anastomotic site.

In Step 530, the method comprises performing angiographic orientation and selecting a fistula location. Choosing the fistula location can be based upon a lack of thrombus or other soft tissue occlusive matter at the vascular location, as well as lack of plaque or calcified matter. Preferably, the fistula location is chosen at a location where the vein is less than or equal to three millimeters apart from the artery. Techniques can be used to image the vein and artery in side-by-side configurations as well as overlapping (i.e. on top of each other in the image) orientations. Rotation of the imaging device 90° can modify the provided image from a side-by-side image to an overlapping image, and back again. In some embodiments, after a fistula location has been selected, a clinician can orient the fluoroscope such that the vein and artery are shown overlapping, such as with the vein on top of the artery. In some embodiments, the clinician can position a fluoroscope or other imaging device at an angle to the patient approximating 35° RAO.

In Step 540, the method comprises placing a vessel-to-vessel guidewire into the vein, such as while the vein and artery are imaged in an overlapping orientation, as described in Step 530 hereabove. A next step comprises placing a needle delivery device over the vessel-to-vessel guidewire and into the vein. The needle delivery device can comprise a marker, as described in FIG. 2 hereabove, such that a clinician can orient the marker toward the artery. The guidewire can be retracted and subsequently, the needle of the needle delivery device can be advanced toward the target wire and toward the artery. In some embodiments, the vessel-to-vessel guidewire can be placed through a dilator.

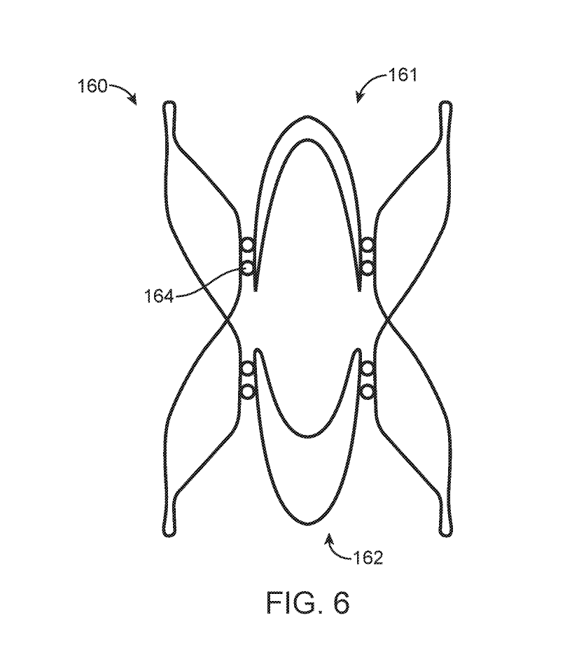

Prior to inserting the crossing needle into the artery, a clinician can aim the needle tip at the center of the artery to ensure desired engagement of the artery with the needle, such as by rotating the proximal end of the needle or a device containing the needle. In some embodiments, the needle or needle delivery device includes a proximal hub with a demarcation (e.g. a flat portion or a marker) positioned to indicate the orientation of a curved distal portion of the needle, such as is described in reference to needle delivery device 140 of FIG. 2 hereabove. In this operation, a clinician can torque or otherwise rotate the needle such that the direction of the needle curvature comes into view on the imaging apparatus (e.g. fluoroscope). Confirming the direction of needle curvature ensures that the needle is to be advanced in the desired direction, such as into the center of the artery. In some embodiments, a target wire is placed in the target vessel, such as target wire 170 of FIG. 2 described hereabove. Preferably, the needle comprises a curved tip, and the radius of curvature can be reduced if a clinician has difficulty in aiming the needle at the center of the target vessel prior to insertion. Conversely, the radius of curvature can be increased to sufficiently aim the needle tip at the center of the target vessel. In some embodiments, the needle delivery catheter is oriented as described in reference to FIG. 6 herebelow.

Additionally, a clinician can confirm that the distal portion of the vessel-to-vessel guidewire is located within the lumen of the artery. Also, the clinician can confirm the vessel-to-vessel guidewire is parallel with the target wire previously placed in the artery. A clinician can confirm that the needle is positioned within the target vessel by using a dye injection through the needle. Alternatively or additionally, a clinician can confirm that the needle is properly positioned in a target vessel by measuring the pressure in a distal portion of the needle, such as to confirm presence in an artery by confirming arterial pressure is recorded.

In some embodiments, the needle delivery device is placed into the artery and the guidewire is advanced from the artery into the vein via the crossing needle. In these embodiments, the anastomotic clip delivery catheter can also be advanced from artery to vein.

In Step 550, the method comprises placing an anastomotic clip at a fistula location. Prior to performing Step 550, placing an anastomotic clip at a fistula location, a user can retract the crossing needle while maintaining the position of the target wire. Next, the target wire can be removed from the second introducer. The target wire can also be removed after Step 550.

In Step 550, a user can position the vein and artery such that the vein and artery are slightly apart from each other on the image (e.g. not overlapping). In one embodiment, this can be achieved by rotating a fluoroscopy unit 45° to 90° after an overlapping image is obtained (e.g. an image obtained during a dual contrast injection of both the artery and vein).

Next, the tip of the clip deployment catheter (with a pre-loaded anastomotic clip) can be placed at the fistula site. In this step, a clinician can apply forward pressure and rotate the clip deployment catheter. The clip can comprise at least two distal arms and at least two proximal arms that can be deployed simultaneously or independently via a control located on the handle of the catheter.

Step 550 further comprises deploying the anastomotic clip in the fistula, such as is described in detail in reference to clip deployment device 150 of FIG. 2 hereabove. The clip distal arms are deployed by moving a control on the clip deployment catheter from a ready to deploy position to a first deployed position, which can be achieved by moving the control relatively parallel to the longitudinal axis of the handle. Prior to deploying the proximal arms of the clip, a clinician can retract the first introducer to the fistula location and seat the vein against the artery. The clip deployment catheter can comprise a marker located on its distal end. Using this marker, a clinician can pull the clip deployment catheter back such that the marker is aligned with the distal end of the first introducer.

In a next operation of STEP 550, the proximal arms can be queued to be deployed via moving the control from a first deployed position to a second ready to deploy position. The ready to deploy position can be achieved by moving the control in a direction perpendicular to the longitudinal axis of the handle. Subsequently, the proximal arms can deployed via moving the control from the second ready to be deployed position to the second deployed position via a motion parallel to the longitudinal axis of the handle. In this embodiment, the control includes a safety position comprising a ready to deploy position which can be transitioned by moving the control in a direction that is perpendicular to the axis of the handle. This control arrangement can prevent inadvertent deployment of the distal and/or proximal arms. After deployment of the proximal arms, a clinician can retract the first introducer from the anastomosis site, such as a retraction of approximately two to three centimeters, followed by retracting the clip deployment catheter.

The method can further comprise dilating the fistula via a balloon or other expandable member. For example, a clinician can track a balloon catheter over the target wire and inflate the balloon. In some embodiments, the balloon catheter comprises a diameter between 1.0 mm and 7.0 mm and can be inflated via a 4 mm diameter by 1.5 cm long non-conforming balloon and indeflator device. The balloon then can be deflated and retracted out of the implant.

The method can further comprise verifying clip patency. This can be achieved via a contrast/saline solution injected into the second introducer. A clinician can then remove all devices once it is confirmed that the clip is positioned as desired.

The method can further comprise placing a second anastomotic clip, such as a second anastomotic clip 160 of FIG. 2 described hereabove. Alternatively or additionally, the method can further comprise creating a second flow pathway between, such as a second fistula created during the same clinical procedure or during a subsequent clinical procedure. The second flow pathway can be between the same two vascular locations as the first flow pathway, or one or both of the second flow pathway vascular locations can be different (e.g. a different vein and/or artery).

The method can further comprise placing a flow restricting element, for example flow restricting element of FIG. 2, within, on and/or proximate the artery or the vein, to at least partially occlude or fully occlude the respective artery or vein. In some embodiments, the flow restricting element can be placed within, on and/or proximate a vein, at a location downstream from the flow pathway. In these embodiments, the flow restricting element can be constructed and arranged to increase flow in a retrograde direction through the vein and into the penis and/or to increase blood pressure in an artery and/or vein of the penis. In some embodiments, the method can include delivering energy to the artery or vein to at least partially occlude or fully occlude the respective artery or vein. For example, the energy can comprise radiofrequency energy delivered to modify tissue (e.g. blood vessel tissue) to restrict flow in a blood vessel.

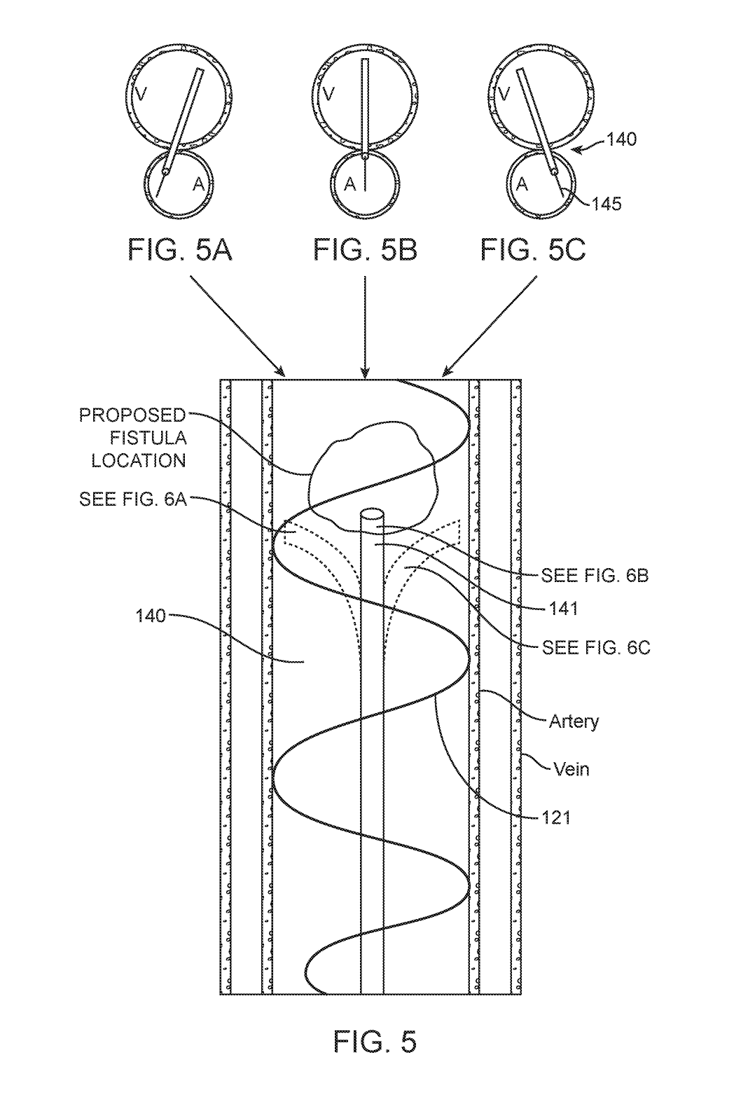

Referring now to FIG. 5, a schematic of an angiographic view of a patient's vein and artery prior to advancement of a needle into the artery is illustrated, such as may be performed in Step 540 of the method of FIG. 4 described hereabove, consistent with the present inventive concepts. In the illustrated embodiment, a clinician has oriented an imaging device (e.g. a fluoroscope or other imaging device of FIG. 1), such that the segments of vein and artery at a proposed fistula location are overlapping (i.e. on top of each other in the image). The clinician has placed a target wire 120 into a patient's artery such that the helical portion of wire 121 is positioned at the proposed fistula location. Additionally, needle deployment device 140 has been advanced intraluminally through the vein as shown such that its distal end is proximal to the proposed fistula location. A next step comprises advancing needle 145 toward the helical portion of wire 121 at the proposed fistula location.

Prior to insertion of needle 145 into the artery, a clinician can rotate needle deployment device 140 such that the direction of curvature of shaft 141 of the needle deployment device 140 is viewed (i.e. a non-linear, curved segment is visualized) on the imaging apparatus. Confirming the direction of curvature ensures that needle 145 is to be advanced in the desired direction, such as into the center of the artery. For example, if a clinician rotates needle deployment device 140 such that its tip is positioned as shown in FIG. 5A or 5C, a clinician will be aiming to an off-center location of the patient's artery. If a clinician rotates needle deployment device 140 such that its tip is positioned as shown in FIG. 5B, needle 145 will subsequently be advanced into the relative center of the patient's artery. The radius of curvature of a needle deployment device 140 can be reduced (e.g. by manual reshaping or by selecting a different needle deployment device 140), such as a reduction in curvature performed when a clinician has difficulty in aiming needle 145 at the center of the artery prior to insertion. Conversely, the radius of curvature of needle deployment device 140 can be increased to create a more desirable needle 145 advancement trajectory.

Referring now to FIG. 6, a perspective view of an anastomotic clip is illustrated, consistent with the present inventive concepts. Clip 160 can comprise at least two distal arms 161 and at least two proximal arms 162. In the illustrated embodiment, clip 160 comprises four distal arms 161 and four proximal arms 162.

Clip 160 can be formed from a single tube of resilient material, such as nickel titanium alloy, spring steel, glass or carbon composites or polymers, or pseudoelastic (at body temperature) material such as nickel titanium alloy or comparable alloys and polymers, by laser cutting several closed-ended slots along the length of the tube (leaving the extreme distal and proximal edges of the tube intact) and cutting open-ended slots from the longitudinal center of the tube through the distal and proximal edges of the tube. The open-ended slots are cut between each pair of closed-end slots to form a number of loops joined at the center section by waist segments. Many other fabrication techniques can be utilized, for example, clip 160 can be made of several loops of wire welded together at the waist section.

After the tube is cut as described above, it is formed into its eventual resiliently expanded configuration. In this configuration, the loops turn radially outwardly from the center section, and evert toward the center plane of the center section, thus forming clinch members, i.e. distal arms 161 and proximal arms 162, in the form of arcuate, everted, petaloid frames at either end of the loop, extending from the generally tubular center section formed by waist segments. For clarity, the term everted is used here to mean that the arc over which the petaloid frame runs is such that the inside surface of clip 160 faces radially outwardly from the cylinder established by the tube.

Once clip 160 has resiliently expanded to the extent possible given its impingement upon the walls of the starting vessel and the target vessel, the center section can be further expanded by plastic deformation. This can be accomplished by inflating a balloon, not shown, within the center section and expanding the center section beyond its elastic or superelastic deformation range. By plastically deforming the center section of clip 160, the center section becomes more rigid and able to withstand the compressive force of the walls of the starting and target vessels.

As illustrated, the construction provides several pairs of longitudinally opposed (that is, they bend to come into close proximity to each other, and perhaps but not necessarily, touch) and aligned (they are disposed along the same longitudinal line) distal arms 161 and proximal arms 162. Overall, the petaloid frames of distal arms 161 form a “corolla,” analogous to the corolla of a flower, flange or rivet clinch, which impinges on the starting vessel wall and prevents expulsion into the target vessel, and the petaloid frames of proximal arms 162 form a corolla, flange or rivet clinch (this clinch would be analogous to a rivet head, but it is formed like the clinch after insertion of the rivet), which impinges on the target vessel wall and prevents the expulsion of clip 160 into the target vessel. Also, the central section forms a short length of rigid tubing to keep the fistula open. The resilient apposition of the at least two distal arms 161 and at least two proximal arms 162 will securely hold clip 160 in place by resiliently clamping the walls of the starting vessel and the target vessel, even over a considerable range of wall thickness or “grip range.”

The respective lengths of arms 161 and 162 can be variably sized to maximize or optimize the stability of clip 160 with respect to the vessels when deployed between adjacent vessels. Moreover, varying the lengths of the respective arms can further provide additional advantages. For instance, the arms which are shortened in length can facilitate the positioning and securement of clip 160 between the vessels by allowing for the relatively shorter member to swing into position within the vessel lumen during deployment, as described in further detail below. Additionally, a shorter member can provide for a minimized implant size when placed against the vessel interior wall for securement as well as a mitigating any physiologic reaction to the implant, e.g., a reduction in thrombosis, etc. Additionally, arms 161 and/or 162 which are lengthened relative to other arms can provide for increased clip stability by increasing the amount of force applied against the tissue walls.

Moreover, arms having different lengths can additionally place the adjacent vessels in tension such that the vessel walls are drawn towards one another and arms 161 and/or 162 contact the vessel luminal walls to stabilize not only clip 160 within the vessels but also the vessels with respect to one another. Additionally, having one or more arms, such as distal arms 161, sized to have a length shorter than its respective apposed clinch member can also facilitate the deployment and/or positioning of distal arms 161 within the vessel, since the shorter length clinch members can more easily “swing” through an arc within the vessel lumen without contacting the interior walls. Arms with differing lengths can further be configured to align along different planes when deployed to facilitate vessel separation, if so desired.

Clip 160 can further comprise at least one marker, not shown, configured to rotationally position the clip at the fistula location. For example, a marker can be oriented toward the target vessel prior to deployment of clip 160. Alternatively or additionally, a marker can be oriented based upon a patient image, e.g. a real-time fluoroscopy image. In yet another embodiment, clip 160 can comprise at least one marker configured to longitudinally position the clip at the fistula location. A marker can indicate the distal and/or proximal end of clip 160.

Clip 160 can further comprise holes 164 configured to engage a clip delivery catheter projection such as to allow the shaft of the clip deployment catheter, not shown, to be retracted while clip 160 remains positioned in the distal portion of the shaft. In one embodiment, holes 164 are constructed and arranged about the clip asymmetrically such that clip 160 can be attached in the proper orientation.

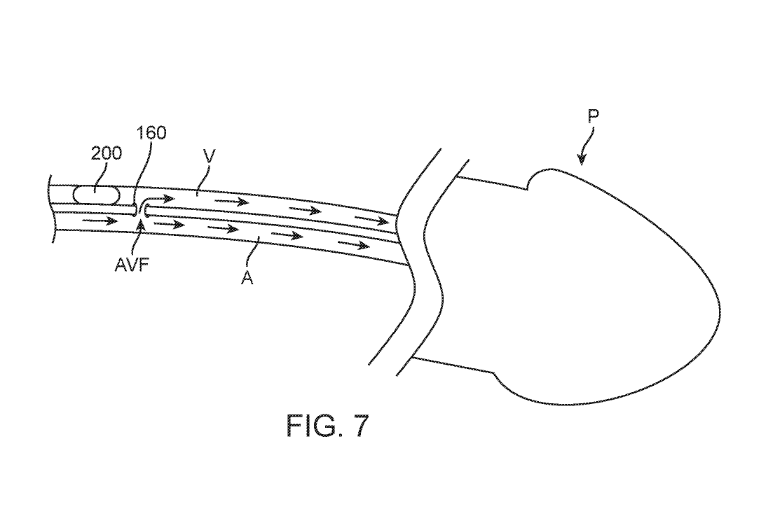

Referring now to FIG. 7, a sectional view of a flow pathway created between a first vascular location and a second vascular location is illustrated, with a flow restricting element implanted within the first vascular location, consistent with the present inventive concepts. Consistent with the systems, devices and methods described herein, a flow pathway can be created between a first vascular location such as a vein, and a second vascular location such as an artery. In the illustrated embodiment, clip 160 is placed between artery A and vein V at fistula location AVF, such as to treat erectile dysfunction. In some embodiments, location AVF is positioned outside of penis P, such as a location proximate to but outside of penis P. In other embodiments, location AVF is selected to be within penis P.

In some embodiments, a flow restricting element can be placed within, on and/or proximate artery A and/or vein V, and/or a separate artery or vein that is fluidly connected to artery A or vein V, respectively. The flow restricting element 200 can be constructed and arranged to modify flow of blood in penis P, such as to treat erectile dysfunction. For example, flow restricting element 200 is placed within vein V as shown in FIG. 7. In some embodiments, flow restricting element 200 can be placed within, on and/or proximate vein V, at a location downstream from the flow pathway located at location AVF. In these embodiments, flow restricting element 200 can be constructed and arranged to increase flow in a retrograde direction through the vein and into the penis and/or to increase blood pressure in an artery and/or vein of the penis. Flow restricting element 200 can comprise an element selected from the group consisting of: a plug such as a self-expanding vascular plug placed within a blood vessel; a stent with a covered portion such as a vascular stent including one or more covered end portions; a clip such as a vascular clip placed around a blood vessel to limit flow; an implantable clamp; an expandable assembly placed proximate a blood vessel to limit flow; a vessel sealant or occluding agent; and combinations of these. In some embodiments, flow restricting element 200 is constructed and arranged as described in applicant's U.S. Pat. No. 8,226,592, entitled “Method of Treating COPD with Artificial Arterio-Venous Fistula and Flow Mediating Systems”, filed Dec. 15, 2004, the contents of which is incorporated herein by reference in its entirety. Flow restricting element 200 can comprise a material selected from the group consisting of: a shape memory material such as nickel titanium alloy; stainless steel; a polymer; a biodegradable material such as magnesium or a bioabsorbable polymer; and combinations of these.

The system, for example system 100 described herein, can further include an energy delivery element or device, not shown but configured to deliver energy to the first or second vascular location to at least partially occlude or fully occlude the respective first or second vascular location. For example, the energy can comprise radiofrequency energy delivered to modify tissue (e.g. blood vessel tissue) to restrict flow in a blood vessel.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

What is claimed is:

1. A method of treating erectile dysfunction in a patient, comprising:
selecting a patient exhibiting erectile dysfunction; and
creating a flow pathway between a first vascular location and a second vascular location;
wherein the first vascular location comprises a source of arterial blood;
wherein the second vascular location comprises a source of venous blood;
wherein the method is constructed and arranged to treat erectile dysfunction.
